(19) United States
(12) Patent Application Publication
(10) Pub. No.: US 2012/0178991 A1
(43) Pub. Date: Jul. 12, 2012

(54) CAMERA, CAMERA SYSTEM, AND METHODS OF USING THE SAME

(75) Inventors: Robert F. Clark, Baldwinsville, NY (US); Vincent A. Primerano, Camillus, NY (US)

(73) Assignee: QuickLook, Inc., Liverpool, NY (US)

(21) Appl. No.: 12/987,205

(22) Filed: Jan. 10, 2011

(51) Int. Cl. A61B 1/04

(52) U.S. Cl. 600/109

(57) ABSTRACT

A camera and a camera system as well as methods of using the camera and the camera system. The camera is very preferably portable and hand-held and includes a wand or probe for insertion into a mouth or other body cavity for the purpose of obtaining an image of regions in such cavity. The images are transmitted onto a display screen for viewing by a doctor or an oral surgeon, by a patient, or by another person. In the preferred embodiment, the camera includes various ways of automatically disabling and enabling operation of the camera, thereby conserving energy.

### FIELD OF THE INVENTION

The present invention generally relates to cameras and camera systems, including methods of using the same. The present invention is especially adapted for use in connection with the mouth and other cavities of a human or other animal. In particular, the present invention in a relatively narrow respect relates to the display of images of the teeth, gums, and other regions of a patient's mouth in connection with dentistry or oral surgery procedures.

### BACKGROUND OF THE INVENTION

Dentists and oral surgeons treat conditions in a patient's mouth, where it is difficult for a patient to see the conditions or to see the dentist's or oral surgeon's treatment of those conditions. Sometimes dentists and oral surgeons use mirrors to help a patient see such conditions and treatment, however, the use of mirrors is awkward and also often fails to reveal in any detail the nature of the conditions or the treatment.

Photographs, such those taken with a digital camera, have been taken of a patient's mouth, however, again, the detail is often lacking and usually several such photographs must be taken at different angles in order to gain a better appreciation of some detail. Further, due to the size of cameras used for taking such photographs, there are many instances where such cameras offer only a limited view of the conditions and the treatment.

Camera systems have been designed that include a probe for insertion into a patient's mouth which receives light and images taken from a wide variety of selected positions. The received images are transmitted to a remote computer for display on a computer screen. Such systems are bulky and expensive, and are not portable from one office to another office. Also, the computer screens that display that the images are usually a significant distance away from the patient, which may pose problems with the patient's viewing of the images displayed on the computer screen.

In the context of this background, the present invention was developed.

### SUMMARY OF THE INVENTION

The present invention relates to a camera and a camera system as well as methods of using the camera and the camera system. The camera is very preferably portable and hand-held and includes a wand or probe for insertion into a mouth or other body cavity for the purpose of obtaining an image of regions in such cavity. The images are transmitted onto a display screen for viewing by a doctor or an oral surgeon, by a patient, or by another person. In the preferred embodiment, the camera includes various ways of automatically disabling and enabling operation of the camera, thereby conserving energy.

### BRIEF DESCRIPTION OF THE DRAWINGS

The present invention will be described with reference to the accompanying drawings wherein:

FIG. 1 is a perspective view from the front of a camera system in accordance with one embodiment of the present invention including a wand or probe resting on the top of a display which in turn is seated in a recharging station;

FIG. 2 is a different perspective view from the rear and the side of the camera system shown in FIG. 1 that more clearly illustrates a conventional electric plug connected by means of a wire cord to the recharging station;

FIG. 3 is yet another perspective view from the rear of the camera system shown in FIG. 1;

FIG. 4 is a perspective view of the rear surface of the recharging station of the camera system shown in FIG. 1;

FIG. 5 is a different perspective view from the front of the recharging station of the camera system shown in FIG. 1;

FIG. 6 is a plan view of the wand or probe connected through a wire cord to the display of the camera system shown in FIG. 1;

FIG. 7 is a perspective view of the bottom of the display of the camera system shown in FIG. 1;

FIG. 8 is a rear plan view of the display shown in FIG. 7;

FIG. 9 is a perspective view of the rear of the display shown in FIG. 7;

FIG. 10 is a perspective view of the wand or probe of the camera system shown in FIG. 1;

FIG. 11 is an exploded view of the wand or probe shown in FIG. 10;

FIG. 12 is an exploded view of the display shown in FIGS. 7-9;

FIG. 13 is an exploded view of the recharging station shown in FIGS. 1-4;

FIG. 14 is a fragmented, perspective view of the wand and probe, the display, and the recharging station shown in FIG. 1; and

FIG. 15 is another perspective view of the camera system shown in FIG. 1.

### DESCRIPTION OF A PREFERRED EMBODIMENT

The present invention will be described with reference to the accompanying drawings wherein like reference numerals referred to the same item.

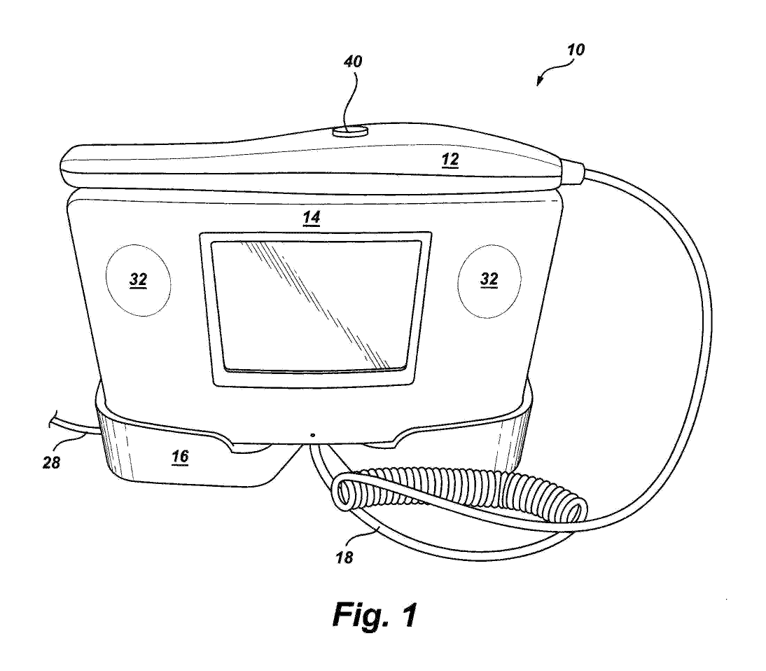

There shown in FIG. 1 a camera system 10 in accordance with a preferred embodiment of the present invention. The camera system 10 includes three principal components: a wand or probe 12, a display 14, and a recharging station 16. The principal components of the camera system 10 are shown in FIG. 1 in a condition of storage or recharging, as will be appreciated from refurther reading this description.

The probe 12 is adapted to rest on the top surface of the display 14. The configuration and surface contours of the outside surface of the probe 12 and the top surface of the display 14 are preferably designed so as to conformingly engage with each other and to maintain the probe 12 seated on the top surface of the display 14. As such, the probe 12 and the top surface of the display 14 preferably possess frictional and other engagement that inhibits the probe 12 from sliding off the top surface of the display 14. The probe 12 is connected to the display 14 by means of an electric wire cord 18. Preferably a portion of the wire cord 18 is coiled in a well-known manner so that the effective length of the cord 18 may be extended against the bias of the coiled portion thereof, and so the coiled portion maintains a bias tending to reduce the effective length of the cord 18.

Preferably each end of the cord 18 possesses a plug adapted to be selectively, connectively received in an associated socket in either the probe 12 or the display 14, respectively. The plugs are adapted to be selectively, releasably received in the associated sockets so that the probe 12, the display 14, and the cord 18 may be selectively disconnected and so that the cord 18 may be replaced.

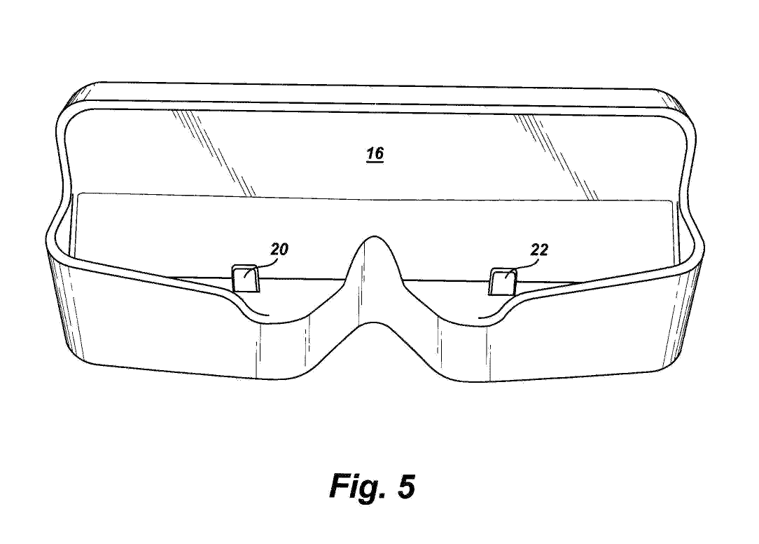

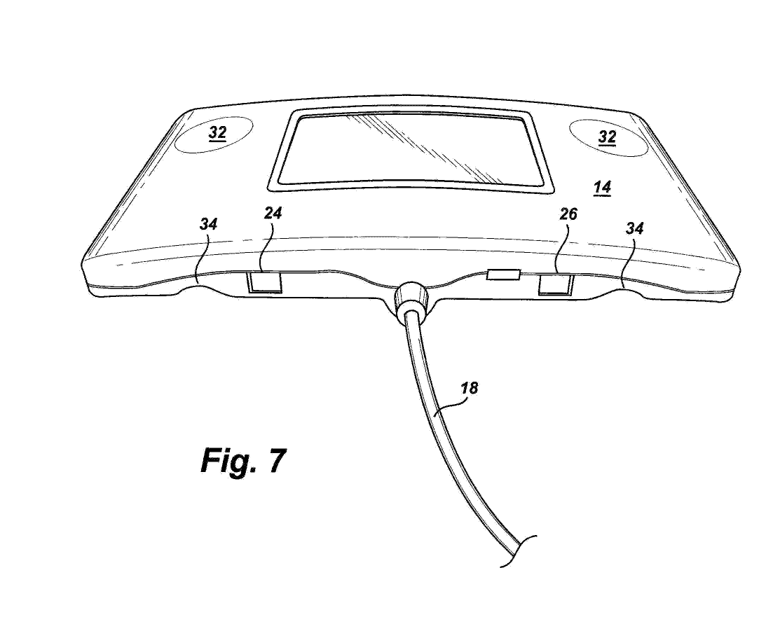

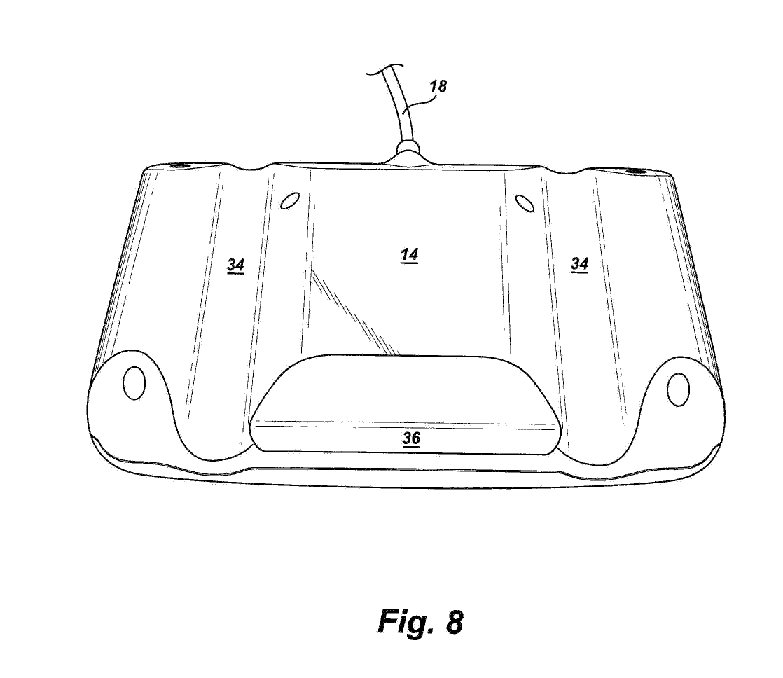

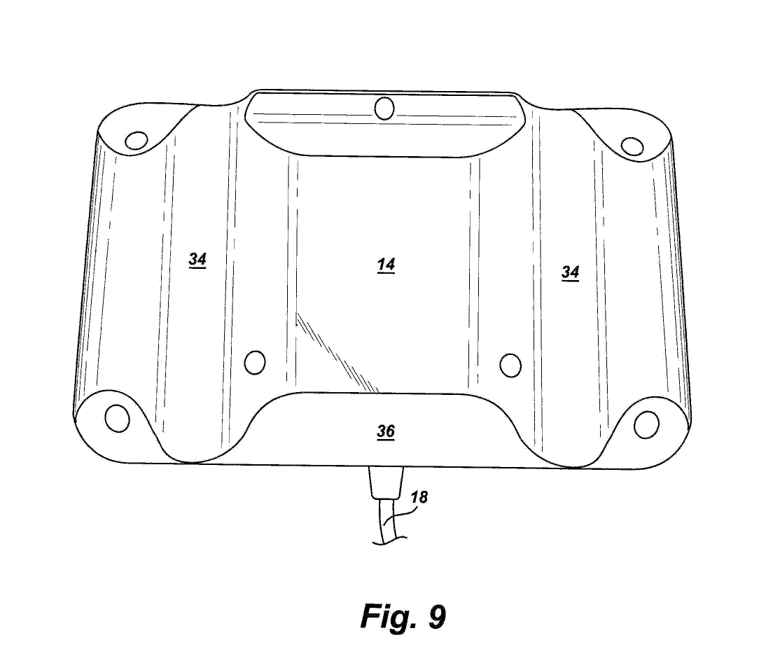

As best shown in FIGS. 5 and 7, the upwardly facing bottom surface of the charging station 16 possesses a pair of spaced, electrical, metal contacts 20, 22 there adapted to operatively contact an associated pair of similarly spaced, electrical, metal contacts 24, 26 disposed in the bottom surface of the display 14. As will be explained in more detail later herein, the electrical contacts 20, 22, 24, and 26 permit the recharging station 16 to recharge batteries disposed within the display 14.

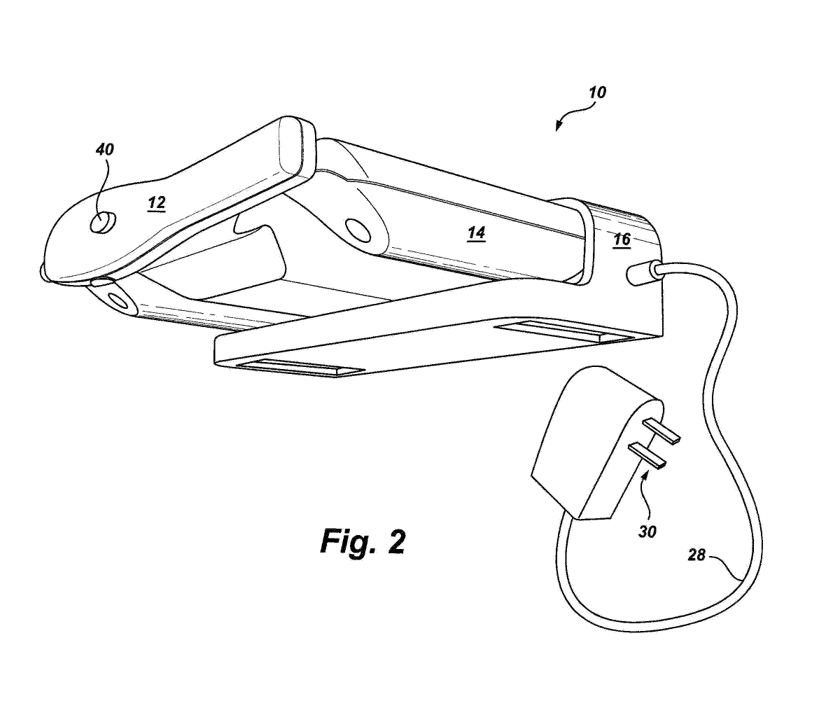

The recharging station 16 may be connected via an electric cord 28 to a conventional electric socket or outlet from which electric power may be obtained to recharge the batteries in the display 14. Accordingly, the cord 28 is provided at an end thereof with a conventional electric plug 30 adapted to be selectively, electrically mated with a conventional electric socket (not shown). The other end of the cord 28 may be provided with a plug adapted to be selectively received in an associated socket in a side of the recharging station 16, as best shown in FIG. 2.

As best shown in FIGS. 1 and 5, the recharging station 16 is provided with a recess or indentation along the front edge thereof so that the electric cord 18 extending between the probe 12 and the display 14 will not be crimped, but may be loosely accommodated, when the display 14 is seated in the recharging station 16. Also as best shown in FIGS. 1 and 5, the recharging station 16 includes a raised back plate extending along the entire length thereof and includes an upwardly extending lip generally along each side thereof and along the front thereof except where the recess is located in the front edge thereof. It will thus be appreciated that the bottom surface of the display 14 preferably conforms with the adjacent, upwardly facing bottom surface of the recharging station 16 whereby that the configuration and contours of the adjacent surfaces substantially conform, thereby providing a relatively stable base upon which the display 14 may rest and helping to insure that the electric, metal contacts 20, 22, 24, and 26 are maintained in a condition of contact. The back plate and the upwardly extending lips of the recharging station 16 help secure the display 14 in such a selected, seated position in the recharging station 16 such that minor, inadvertent bumps against the display 14 or the recharging station 16 will not dislodge the display 14 from the recharging station 16 and such that purposeful, manual removal of the display 14 from the recharging station 16 is easily facilitated.

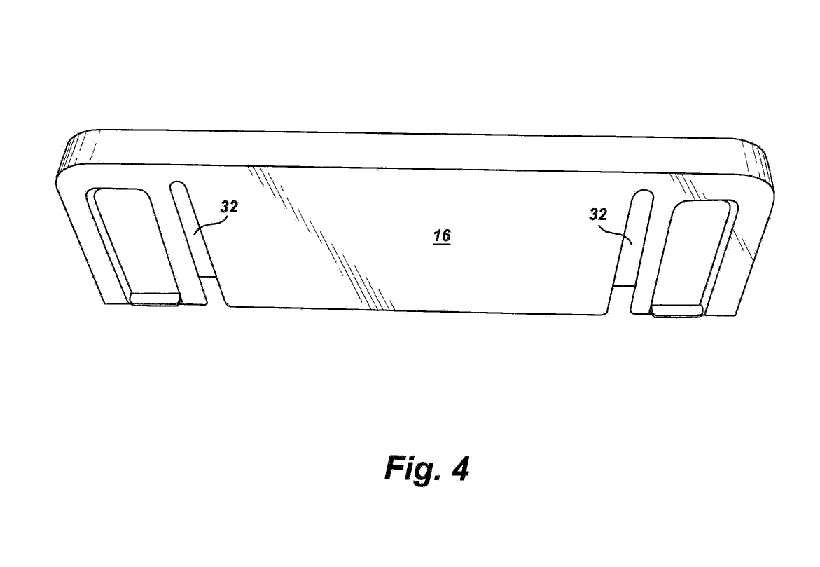

As best shown in FIGS. 2-4, the rear surface of the back plate of the recharging station 16 is provided with a pair of spaced, parallel grooves or slots 32. The upper end of each slot may be recessed so as to accommodate the head of a screw or nail or other mounting implement mounted or otherwise attached to a wall or similar structure. As such, the recharging station 16 may be selectively mounted on a wall or similar structure by means of the slots 32. The invention also contemplates that the width of the slots 32 may be tapered toward their upper ends so as to accommodate different sizes of the heads of nails, screws, or the like.

The display 14 includes a rectangular liquid crystal display (“LCD”) screen mounted on front face thereof that is adapted to display images of the patient's mouth, including images of teeth, gums, and other features of the mouth. Each side of the front surface of the display 14 includes a dimple 34 or depression as best shown in FIGS. 1, 6, and 7 adapted to receive a corresponding thumb of the user of the camera system 10. Such user may be the dentist or oral surgeon or may be the patient, for example. The rear surface of the display 14 includes a pair of spaced, parallel channels 36 or recessions disposed near an associated side of the display 14. The channels 36 are adapted to receive the fingers of corresponding hands of the user of the camera system 10. A top, rear surface of the display 14 includes a recessed region 36 so that a user of the camera system 10 may easily grasp the display 14 in the region of the recess 36 while also clasping the probe 12 against the top surface of the display 14, thereby permitting a user of the camera system 10 to easily transport the display 14 together with the probe 12. It will be appreciated that when the display 14 and the probe 12 are so carried together, the coiled cord 18 assumes a relatively retracted length, thereby minimizing the possibility that the cord 18 may become snagged, cause the user to trip, or otherwise interfere with the advantageous use of the camera system 10.

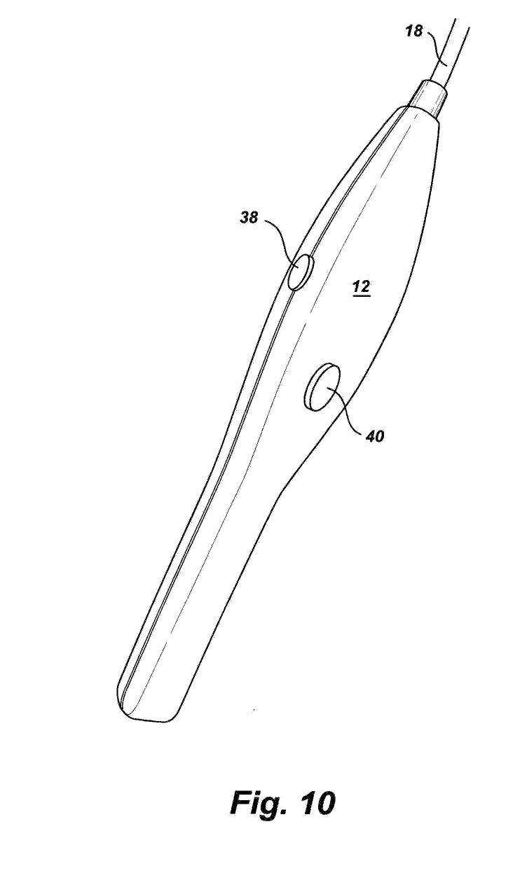

As is best shown in FIGS. 1-3 and 10, the probe 12 includes a bulbous handle portion and an elongated end extending from the bulbous handle portion and adapted to be inserted into the patient's mouth or other cavity. In order to ensure sanitary use of the probe 12, a thin prophylactic plastic disposable sheath (not shown) is adapted to be selectively, disposably mounted over the elongated end portion of the probe 12 that is adapted to extend into the patient's mouth or other cavity. Preferably the sheath is transparent so as to transmit light without significant reflection or absorption, or at least a portion of the sheath is of such a character, which portion would be adapted to be disposed over a lens mounted in the elongated tip of the probe 12, as will be explained in more detail below. Preferably such lens is located in the tip of the probe 12 so that the lens is disposed facing downwardly toward the top of the display 14, in a protected position, when the probe 12 and the display 14 are placed adjacent to each other, such as a position shown in FIG. 1.

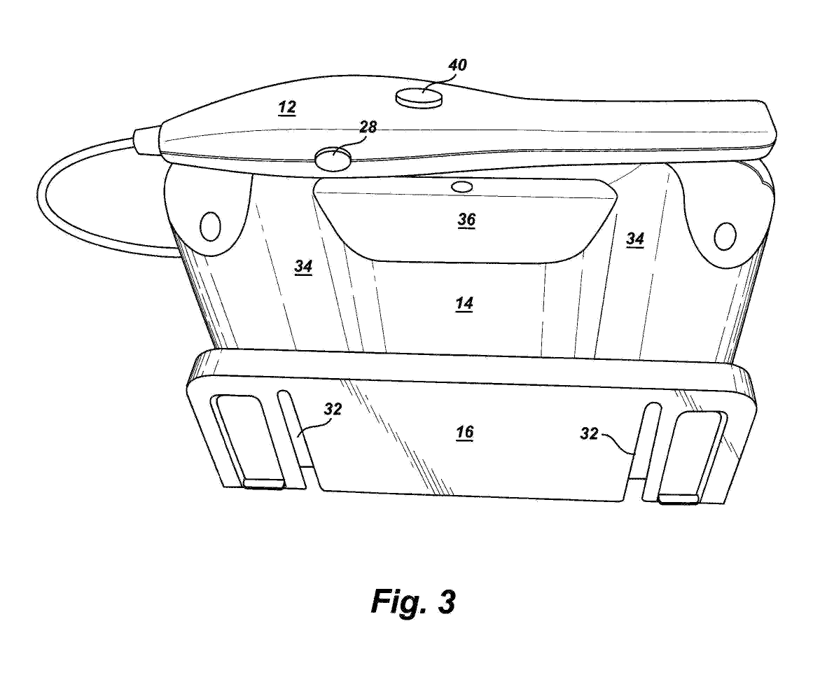

It will also be appreciated from reviewing FIG. 3 that the probe 12 includes a pair of manually depressible buttons 38, 40. The button 38 may be used to freeze or unfreeze an image displayed on the display screen of the display 14. The button 40 actuates a zoom feature associated with the lens. As such, the button 40 may be a toggle type button that may be selectively manipulated to zoom in and out. The zoom feature may be either an optical zoom, in which the distance between the lens and an image sensor that receives light transmitted by the lens is changed, or may be a digital zoom, in which software technology crops and magnifies the digital image to create an artificial close-up image. The latter, digital zoom requires magnifying or removing individual pixels, which can cause image quality degradation. Consequently, the present invention preferably utilizes an optical zoom to provide changes in magnification of the image. It should be appreciated that such zoom feature is not a necessary aspect of the present invention.

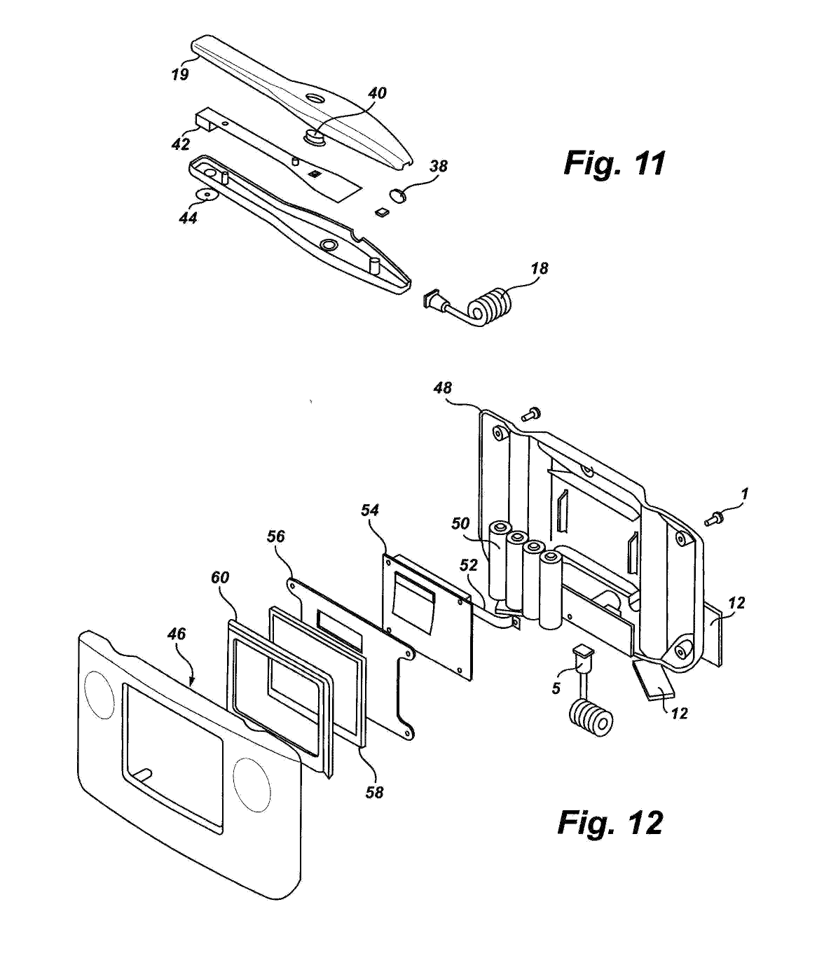

FIG. 11 shows an exploded, detailed view of the components of the probe 12, which generally includes a plastic housing formed of two halves or shells which may be secured together by adhesive, screws, bolts, snap-fit members, or other means. One of the shells, or both, may be fashioned of a transparent material. Interposed between and within the shells is a camera sensor board 42 in which the sensor is disposed toward the tip of the probe 12, adjacent to and facing a lens 44 mounted in the wall of the tip of one of the shells of the probe 12. As used herein, the term “lens” is relatively broad, and may include, for example, contoured glass or plastic, such as those having a convex or concave surface, may include plate glass or plastic having parallel planar surfaces, and may simply include a gap or aperture such as is utilized with a “pinhole” camera.

FIG. 12 shows an exploded view of the components of the display 14. The components include a plastic front plate 46 and a plastic back plate 48 that may be secured together by adhesive, screws, bolts, snap-fit members, and other means. The display 14 preferably includes four rechargeable batteries 50 connected by means of a poly ribbon 52 to an LCD board 54. A plastic backer 56 provides a backing to an LCD screen 58, which is mounted in a LCD frame 60, which in turn is mounted in a rectangular aperture in the front plate 46 of the display 16. The LCD board 54 electrically communicates with the LCD screen 58 to control the images displayed on the LCD screen 58.

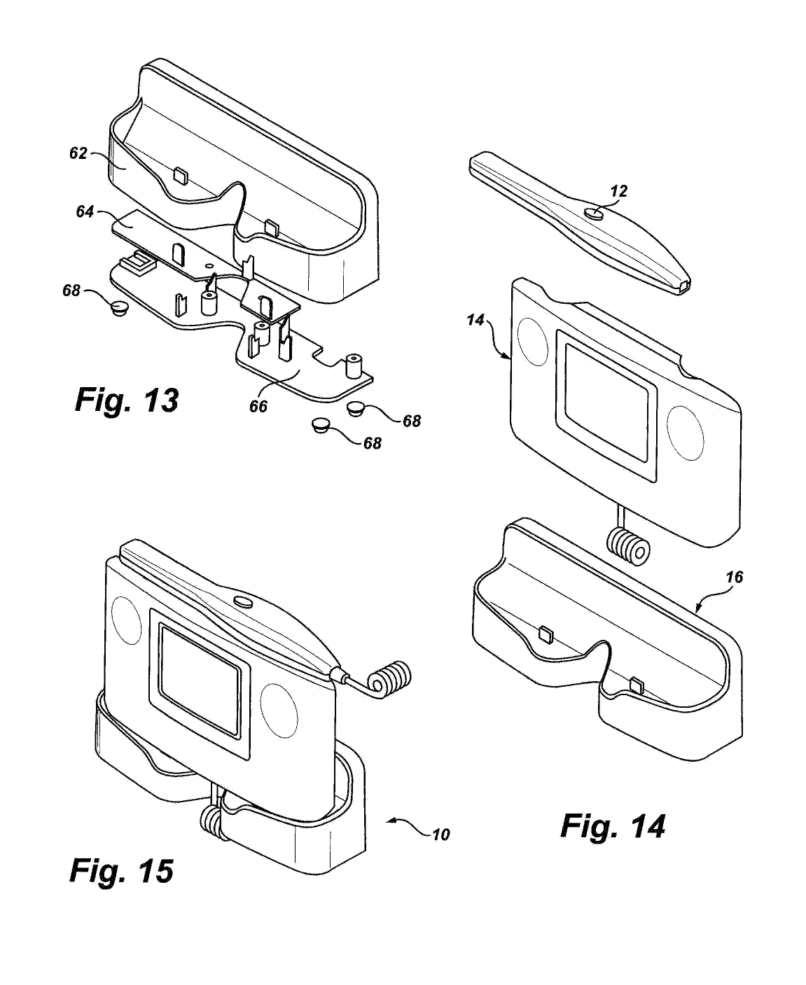

FIG. 13 shows an exploded view of the recharging station 16, which includes a base 62, a charging board 64, and a base cover 66. Optionally mounted on the lower surface of the base cover 66 are a plurality of feet 68 that may be fashioned of rubber, felt, wood, metal, ceramic, or other material and that may be secured to the base cover 66 by adhesive or other well-known means. It will be appreciated that the feet 66 help to prevent the recharging station 16 from sliding and potentially marring a counter top or other supporting surface (not shown).

FIG. 14 shows the basic components of the probe 12, the display 14, and the recharging station 16. FIG. 15 shows the probe 12, the display 14, and the recharging station 16 in a state of storage and general inactivity, much like the camera system 10 shown in FIG. 1.

The camera system 10 of a preferred embodiment of the present invention generally operates as follows. The dentist or oral surgeon removes the probe 12 and the display 14 from the recharging station 16 by grasping the display 14 in the region of the recess 36 on the top region of the back surface of the display 14. In such a condition, the batteries 50 are normally in a fully charged state. The dentist or oral surgeon then places a transparent plastic sheath over the extended end of the probe 12 and then inserts the probe 12 into the mouth of a patient. The dentist or oral surgeon or the patient may grasp the display 14 with either one hand or both hands such that the person's thumbs are disposed in the dimples 34 on the front face of the display 14 and the person's fingers are disposed in corresponding ones of the channels 36. The person then maneuvers the display 14 so that the LCD screen 58 is viewable by either the doctor or oral surgeon or the patient, or both. Light received by and transmitted through the lens 44 is received by the camera sensor board 42, which transforms the image into an electrical signal which is transmitted through the coiled cord 18 to the display 14, and in particular, to the LCD board 54, which in turn converts the electrical signal to recreate the image on the LCD screen 58. The LCD display screen 58 will display the images received by the camera sensor board 42 in real-time. By selectively depressing the freeze button 38, a still image may be maintained on the LCD screen 58.

It should be appreciated that instead of a wired connection via the coiled cord 18 between the probe 12 and the display 14, the probe 12 could be provided with its own batteries or other power source whereby the electrical signals from the camera sensor board 42 could be transmitted wirelessly to the display 14.

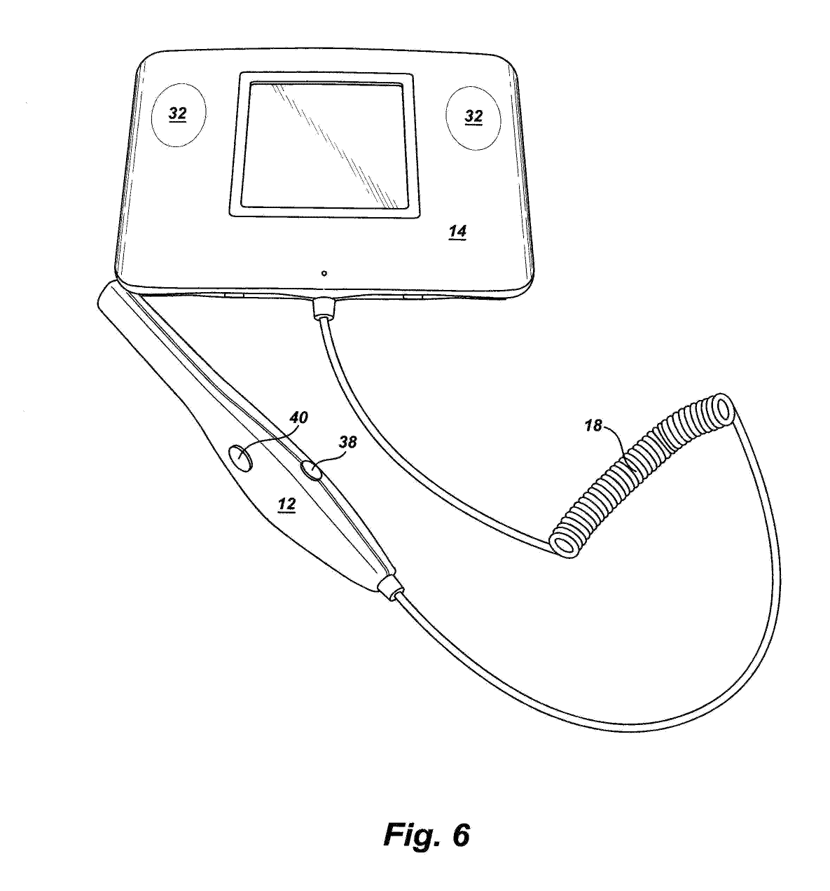

The display 14 is preferably made automatically operational/enabled or non-operational/disabled by a tilt sensor switch (not shown) that detects a relative orientation of the display 14. For example, the tilt sensor switch may be constructed or programmed to recognize that orientation of the display 14 when it is lying on its back surface on a horizontal table top, such as is shown in FIG. 6, constitutes a “horizontal” orientation. One such tilt sensor switch is manufactured by OncQue P/N RBS070500T. The switch comprises essentially a ball bearing which touches two electrical contacts when positioned upright, thereby producing a closed circuit. When the switch is horizontal, the ball bearing rolls away from the two electrical contacts and produces an open circuit. The switch includes a pull-up resistor connected to one contact, and the other contact is connected to ground. The open circuit produces a logic 1 (indicating horizontal position), and the closed circuit produces a logic 0 (indicating not in horizontal position).

If someone picks up the display 14 from a countertop or the like and the orientation of the display 14 changes to more than a preselected amount, such as more than twenty degrees relative to the defined “horizontal”, then the tilt sensor switch will activate the display 14 such that images received by the camera sensor board 42 are displayed on the LCD screen 58. It will be appreciated that any orientation may be the defined standard orientation and that a wide variety of deviations from that standard may be selected as the deviation from that standard that provides a threshold for triggering the enablement of the display 14. For example, the tilt sensor switch may be constructed or programmed to enable the display 14 when the display 14 is oriented more than fifteen degrees from the “horizontal” or more than thirty degrees from the “horizontal”. The invention also contemplates that the standard orientation from which a deviation is monitored, instead of being substantially a true horizontal of a plane extending generally midway between the front plate 46 and the back plate 48 of the display 14, may be any selected orientation of the display 14. Preferably, the standard orientation is substantially horizontal since the LCD screen 58 and the display 14 will assume a substantially vertical orientation when being viewed, and then the doctor or oral surgeon will lay the display 14 flat on its back surface on a counter top or shelf when temporarily not in use.

The invention also contemplates that the display 14 may include a timer circuit used in conjunction with the tilt sensor switch such that either (a) the display 14 will not be activated unless the display 14 is in an orientation exceeding a predetermined range away from “horizontal”, such as more than twenty degrees from horizontal, and is maintained at such orientation for longer than a predetermined time, or (b) the display 14 will be maintained in an activated mode for a certain preselected amount of time after it has been activated, regardless of whether the display 14 returns to an orientation that is within a certain range of “horizontal”, such as less than twenty degrees from horizontal. Each of these time intervals may be selectively chosen.

Once the display 14 has been activated, then the tilt sensor switch may be used to deactivate the display 14 whenever the display 14 attains a preselected orientation such as less than twenty degrees from the “horizontal”. However, preferably the display 14 is deactivated only when the display 14 has maintained such orientation for a preselected time duration. Such automatic deactivation saves energy and minimizes the display of unwanted or irrelevant images.

It should be appreciated that the display 14 may be automatically activated whenever the display 14 attains an orientation outside a predetermined range from “horizontal” or some other predefined orientation, but preferably only when the device 14 has maintained such orientation for a preselected time duration. The display 14 may also be automatically deactivated by a reverse process. If the display 14 is activated and the display 14 attains an orientation within the preselected range from “horizontal” or some other defined orientation and the display 14 has attained such orientation for a preselected amount of time, then the display 14 will be automatically deactivated so that images are no longer displayed on the LCD screen 58.

Typically, the time intervals for the time duration for activating or deactivating the display 14 when the display 14 has attained an orientation within the preselected range from standard orientation or outside the preselected range from standard orientation may be selected to be anywhere from about two seconds to two minutes, and preferably about five seconds to ten seconds. It should be understood that the selected duration of time for activating the display 14 may be different from the selected duration of time for deactivating the display.

The invention also contemplates that the display 14 may have a master on/off switch (not shown) that overrides the foregoing activation and deactivation features, or that may be used as an alternative to such automatic activation and deactivation. It will be appreciated that a controller in the display 14, such as the LCD board 54, may be operatively interconnected with the tilt sensor switch and with the timer circuit as so to implement the automatic activation and deactivation.

A controller in the display 14, such as the LCD board 54, may also be utilized to regulate the charging of the rechargeable batteries 50. The controller may sense the presence of a charging voltage from the charging station 16 transmitted through the electric, metal contacts 20, 22, 24, and 26. If the controller senses such a voltage, then the controller may automatically turn off, that is, deactivate, the display 14 so that no images are displayed on the LCD screen 58 while the display 14 is seated in the charging station 14 and being recharged. The controller then may turn on a charging P-FET transistor that routes a trickle charging current from the recharging station 16 to the rechargeable batteries 50. The controller then continues to monitor the battery voltage and also preferably monitors the amount of time that the batteries are being recharged by electrical connection with the recharging station 16. The controller may terminate the recharging of the batteries when the battery voltage is detected to be greater than a predetermined value such as if the voltage exceeds 5.3 volts or when the total time recharging the batteries is greater than a preselected amount, such as five hours. Either or both the voltage threshold and the time threshold may be selectively varied and may be programmed into the controller.

The preferred camera system has three states of operation: “in-use”, “not-in-use”, and “battery-charge”. The system utilizes the tilt sensor to determine if the handheld unit is upright and “in-use” versus the horizontal position which is presumed to be the “not-in-use” state. The preferred camera system processes logic and continuously monitors the battery charge voltage contacts. If the voltage on the battery charge contacts is greater than a prescribed voltage, then the system processing goes into the “battery-charge” state and ignores the tilt sensor signal. If not in a “battery-charge” state, the processing continuously monitors the tilt sensor signal. If the tilt sensor signal is a logic 0 (not horizontal), the processing sets the state to “in-use” and turns on a FET switch thereby powering the LCD display in the display 14 and the inter-oral camera in the probe 12. If the tilt sensor signal is a logic 1 (horizontal) and the system is currently in the “in-use” state, the processing counts the duration of time that the display 14 is lying in the horizontal position. Once a prescribed amount of time is reached, the processing turns off the LCD display power, turns off the inter-oral camera power, sets the state to “not-in-use”, and sets the microprocessor to a low current sleep mode where no processing is performed in order to conserve battery life.

If the display 14 is taken out of the horizontal position before the prescribed amount of time, the time count is reset, and the system stays in the “in-use” state. If the system is in the “not-in-use” state, a transition of the tilt sensor signal from a logic 1 to a logic 0 wakes up the microprocessor, sets the “in-use” state, and resumes the processing loop of continuously monitoring the tilt sensor signal and the battery charge voltage contacts to determine which state to be in. This transition from a logic 1 to a logic 0 is the only way that the microprocessor transitions to the “in-use” state and resumes processing in the preferred embodiment.

The present invention additionally contemplates that the tip of the probe 12 may be provided with a light that is selectively activated so as to help illuminate a patient's mouth or other cavity. Also, many of the preselected values or features may be implemented during fabrication of the camera system 10 so as to be effectively fixed during use thereafter, or may be variably selected after fabrication by a customer or user of the camera system 10 such as by providing adjustment knobs on the outside surface of the display 14 or by means of a touch screen on the display 14.

While the invention has been described with reference to preferred embodiments, it will be understood by those skilled in the art that various changes may be made and equivalents may be substituted for elements thereof to adapt to particular situation without departing from the scope of the invention. Therefore, it is intended that the invention not be limited to the particular embodiments disclosed as the best mode contemplated for carrying out this invention, but that the invention will include all embodiments falling within the scope and spirit of the appended claims.

1. A camera system adapted for displaying images of a mouth or other cavity of a human or other animal, said camera comprising:
(a) a wand adapted to be hand-held and portable and at least of portion of which is adapted to be inserted into the cavity, said wand including
a lens adapted to receive light from the cavity and to transmit the received light; and
a camera sensor adapted to receive light transmitted through said lens in the form of an image and to transform the image received thereon into a corresponding electronic signal;
(b) a display adapted to be hand-held and portable, said display including: a display screen adapted to be viewed by a user of the camera system; and
a controller operatively coupled to said camera sensor and to said display screen and adapted to receive said electronic signal from said camera sensor and to transform said electronic signal such that the image received on said camera sensor is recreated on said display screen;
(c) means for transmitting said electronic signal from said camera sensor to said controller;
(d) a source of electric power adapted to be operatively connected to at least one of said camera sensor and said controller; and
(e) means for operatively disconnecting said electric power source from at least one of said camera sensor and said controller such that an image received on said camera sensor is not recreated on said display screen, said disconnecting means responsive to a preselected range of orientation of said display and a time duration in which said display is in said orientation range.

2. A camera system according to claim 1 further comprising:
(f) means for operatively connecting said electric source of power to at least one of said camera sensor and said controller such that an image received on said camera sensor is recreated on said display screen, said connecting means responsive to a different range of orientation of said display.

3. A camera system according to claim 1 wherein said disconnecting means includes a tilt sensor and a timer.

4. A camera system according to claim 2 wherein said connecting means includes a tilt sensor.

5. A camera system according to claim 1 further comprising either an optical zoom or a digital zoom by which the magnification of the image received on said camera sensor may be selectively changed.

6. A camera system according to claim 1 wherein said disconnecting means is adapted to be selectively modifiable by a user of said camera by selectively changing at least one of said orientation range or said time duration.

7. A camera system according to claim 1 further comprising an image freeze electronic circuit operatively connected to said controller to cause said controller to selectively maintain a recreated image on said display screen.

8. A camera system according to claim 7 wherein said circuit includes a manually actuated electric switch.

9. A camera system according to claim 1 wherein said electric power source includes at least one rechargeable battery.

10. A camera system according to claim 1 further comprising a source of light operatively mounted to said wand and adapted to illuminate the cavity.

11. A camera system according to claim 1 where said display possesses a defined horizontal position and wherein said orientation range essentially consists of a range of degrees from said horizontal position.

12. A camera system according to claim 11 wherein said range of degrees from said horizontal position is about plus or minus twenty degrees from said horizontal position.

13. A camera system according to claim 1 wherein said time duration is within the range of about five to sixty seconds.

14. A camera system according to claim 9 further comprising a charging station adapted to mountingly receive said display, said charging station and said display each possessing a cooperating portion of an electric connector, said cooperating portion possessed by said charging station adapted to be in operative connection with a electric power source external to said camera system and said cooperating portion possessed by said display operatively connected to said at least one rechargeable battery, such that said at least one rechargeable battery may be recharged from said external electric power source, said camera system further comprising a voltage sensor for monitoring the voltage of said at least one rechargeable battery and a charging timer for monitoring the amount of time said at least one rechargeable battery has been recharged by said external electric power source, and wherein said camera system further comprises a regulator responsive to said voltage sensor and to said charging timer and adapted to terminate the recharging of said at least one rechargeable battery if the voltage of said at least one rechargeable battery exceeds a predetermined threshold voltage and if the amount of time said at least one rechargeable battery exceeds a predetermined threshold amount of time.

15. A method of creating an image of a mouth or other cavity of a human or other animal comprising:
(a) providing a camera adapted to be hand-held and portable, said camera including a probe adapted to be inserted at least partially into the cavity and to receive images from the cavity and a display adapted to display to a user of the camera the images received by said probe;
(b) inserting said probe at least partially into the cavity such that images of the cavity are received by said probe and displayed by said display; and
(c) automatically disabling said camera from displaying the images received by said probe if said display assumes an orientation within a predetermined range of orientations for a predetermined time duration.

16. A method according to claim 15 wherein said camera further includes at least one rechargeable battery adapted to provide power for operating said camera, said method further comprising:
recharging said at least one rechargeable battery;
terminating the recharging if the voltage of said at least one rechargeable battery exceeds a predetermined threshold and if the duration of the recharging exceeds a predetermined amount of time.

17. A method according to claim 15 further comprising:
disabling said camera from displaying the images received by said probe; and
after said camera system has been disabled, re-enabling said camera to display the images received by said probe if said display assumes an orientation outside of said predetermined range of orientations.

18. A method according to claim 15 further comprising:
selectively maintaining a still display of an image.

19. A method of creating an image of a mouth or other cavity of a human or other animal comprising:
(a) providing a camera adapted to be hand-held and portable, said camera including a probe adapted to be inserted at least partially into the cavity and to receive images from the cavity and a display adapted to display to a user of the camera the images received by said probe;
(b) inserting said probe at least partially into the cavity such that images of the cavity are received by said probe and displayed by said display; and
(c) step for automatically disabling said camera.

20. A method of creating an image of a mouth or other cavity of a human or other animal comprising:
(a) providing a camera adapted to be hand-held and portable, said camera including a probe adapted to be inserted at least partially into the cavity and to receive images from the cavity and a display adapted to display to a user of the camera the images received by said probe;
(b) inserting said probe at least partially into the cavity such that images of the cavity are received by said probe and displayed by said display; and
(c) step for automatically enabling said camera after said camera has been disabled.

21. A portable camera adapted to be hand-held and adapted for displaying images of a mouth or other cavity of a human or other animal, said camera comprising:
(a) a probe adapted to be portable and hand-held and adapted to be inserted at least partially into the cavity and to receive images from the cavity;
(b) a display adapted to be portable and hand-held and adapted to display the images received by said probe; and
(c) means for automatically disabling said camera.

22. A portable camera adapted to be hand-held and adapted for displaying images of a mouth or other cavity of a human or other animal, said camera comprising:
(a) a probe adapted to be portable and hand-held and adapted to be inserted at least partially into the cavity and to receive images from the cavity;
(b) a display adapted to be portable and hand-held and adapted to display the images received by said probe; and
(c) means for automatically enabling said camera after said camera has been disabled.
